(19) United States
(12) Patent Application Publication
(10) Pub. No.: US 2017/0027845 A1
(43) Pub. Date: Feb. 2, 2017

(54) COMPOSITION COMPRISING A SPECIFIC ACRYLIC COPOLYMER, A SOLVENT AND A SPECIFIC PROPELLANT

(71) Applicant: L'OREAL, Paris (FR)

(72) Inventor: Christophe JACQUES, Herblay (FR)

(21) Appl. No.: 15/106,008

(22) PCT Filed: Dec. 19, 2014
(86) PCT No.: PCT/FR2014/053456

§ 371 Date: Jun. 17, 2016

(30) Foreign Application Priority Data
Dec. 20, 2013 (FR) 1363190

(51) Int. Cl. A61K 8/81; B65D 83/14; A61K 8/04; A61Q 5/06; A61K 8/31

(52) U.S. Cl. CPC A61K 8/8158; A61Q 5/06; A61K 8/315; A61K 8/046; B65D 83/752; A61K 2800/87; A61K 2800/95

(57) ABSTRACT

The present invention relates to a composition intended to be applied from an aerosol device, comprising:
(i) one or more acrylic copolymers comprising, as monomers:
   (a) at least one unsaturated acid monomer chosen from acrylic acid, methacrylic acid or their salts;
   (b) at least one unsaturated ester monomer of acrylate or methacrylate type having, in its structure, at least one alkyl group having a fatty chain which is linear or branched, preferably having a C₈-C₃₀, more preferably C₁₀-C₁₅ and better still C₁₂-C₁₃ alkyl group;
   (c) at least one unsaturated ester monomer of acrylate or methacrylate type having, in its structure, at least one alkyl group having a short chain which is linear or branched, preferably branched, preferably having a C₂-C₆, more preferably C₃-C₅ and better still C₄ alkyl group; and
   (d) at least one acrylamide monomer having a linear or branched, preferably branched, alkyl group, preferably having a C₃-C₁₀, preferably C₄-C₈, alkyl group;
(ii) one or more solvents;
(iii) a propellant comprising 1,1-difluoroethane.

The present invention relates to a composition for the treatment of keratinous fibres, in particular human keratinous fibres, such as the hair, intended to be applied from an aerosol device, comprising one or more specific acrylic copolymers, one or more solvents and a specific propellant. The present invention also relates to a method for the shaping and/or form retention of the hairstyle employing the said composition and also to the use of the said composition for the shaping and/or form retention of the hairstyle. The invention also relates to an aerosol device containing the said composition.

In the field of styling, the use of aerosol sprays or foams is well known. They are formulations packaged in aerosol devices which, at the outlet of the aerosol device, are, after spraying, in the form of fine droplets, thus forming a spray, or in the form of an expanded system constituting a foam.

These formulations, generally comprising a fixing polymer, are used to structure the hairstyle and to provide it with lasting hold.

However, certain fixing polymers result in hardening of the head of hair. This disadvantage results in a rigid hairstyle and in disentangling which is often difficult at the end of the day, the hair exhibiting a dry feel.

The documents JP2006-199663 and JP2007-217314 describe hair compositions comprising a film-forming acrylic copolymer, the compositions being packaged in a container pressurized with a mixture of alkanes or with dimethyl ether. The composition described in the document JP2006-199663 makes it possible to obtain good properties of fixing the hair in combination with properties which are non-tacky and which do not generate residues. The composition described in the document JP2007-217314 makes it possible to give a natural rendering without generating tackiness.

However, these propellants are flammable and for this reason exhibit limitations in their uses. Furthermore, the properties obtained in terms of fixing (level and durability) and of cosmetic quality (smooth feel, disentangling) are not optimum.

There thus exists a real need to formulate a lasting high-fixing styling composition which contributes a cosmetic feel to the hair and which can be used without limitations.

The Applicant Company has found, surprisingly and unexpectedly, that the combination of a specific acrylic copolymer, of a solvent and of a specific propellant makes it possible to obtain a head of hair which does not exhibit the above disadvantages.

Specifically, this combination makes it possible to obtain strong fixing of the head of hair, which lasts throughout the day, while contributing a cosmetic feel to the hair. The surface of the head of hair is smoother and the hair is retained in the form desired without being rigidified or hardened.

In addition, the disentangling at the end of the day is facilitated, the feel is non-tacky and the softer hair is supple and smooth.

A subject-matter of the invention is thus a composition intended to be applied from an aerosol device, comprising:
(i) one or more acrylic copolymers comprising, as monomers:
   (a) at least one unsaturated acid monomer chosen from acrylic acid, methacrylic acid or their salts;
   (b) at least one unsaturated ester monomer of acrylate or methacrylate type having, in its structure, at least one alkyl group having a fatty chain which is linear or branched, preferably having a C₈-C₃₀, more preferably C₁₀-C₁₈ and better still C₁₂-C₁₃ alkyl group;
   (c) at least one unsaturated ester monomer of acrylate or methacrylate type having, in its structure, at least one alkyl group having a short chain which is linear or branched, preferably branched, preferably having a C₂-C₆, more preferably C₃-C₅ and better still C₄ alkyl group; and
   (d) at least one acrylamide monomer having a linear or branched, preferably branched, alkyl group, preferably having a C₃-C₁₀, preferably C₄-C₈, alkyl group;
(ii) one or more solvents;
(iii) a propellant comprising 1,1-difluoroethane.

Another subject-matter of the present invention consists of a method for the shaping and/or form retention of the hairstyle employing the composition according to the invention.

A further subject-matter of the invention is the use of the composition according to the invention for the shaping and/or form retention of the hairstyle.

Another subject-matter of the invention is an aerosol device comprising a pressurized container containing a composition according to the invention surmounted by an actuator.

Other subject-matters, characteristics, aspects and advantages of the invention will become even more clearly apparent on reading the description and the example which follow.

As indicated above, the composition according to the invention comprises one or more acrylic copolymers capable of being obtained by copolymerization of the following monomers:
(a) at least one unsaturated acid monomer chosen from acrylic acid, methacrylic acid or their salts;
(b) at least one unsaturated ester monomer of acrylate or methacrylate type having, in its structure, at least one alkyl group having a fatty chain;
(c) at least one unsaturated ester monomer of acrylate or methacrylate type having, in its structure, a short-chain alkyl group; and
(d) at least one acrylamide monomer substituted by at least one alkyl group.

The acrylic copolymer is preferably film-forming.

The term “film-forming polymer” is understood to mean a polymer capable of forming, by itself alone or in the presence of an auxiliary film-forming agent, a macroscopically continuous film on a support, in particular on keratinous substances, and preferably a cohesive film.

The acrylic copolymer comprises at least one monomer chosen from acrylic acid, methacrylic acid or their salts.

Preferably, the acrylic acid, the methacrylic acid or their salts are present in a content ranging from 2 to 10% by weight, preferably from 5 to 9% by weight, with respect to the total weight of the copolymer.

The acrylic copolymer also comprises one or more acrylate or methacrylate unsaturated ester monomers having, in its or their structure, an alkyl group having a fatty chain. These monomers are thus esters of acrylic or methacrylic acid and of a monoalcohol of fatty alcohol type, the monoalcohol providing the alkyl group of the ester. The alkyl group can be linear or branched. Preferably, the unsaturated ester monomer of acrylate or methacrylate type having, in its structure, an alkyl group having a fatty chain is an alkyl acrylate or methacrylate with a C₈-C₃₀, more preferably C₁₀-C₁₅ and better still C₁₂-C₁₃ alkyl group.

Preferably, the unsaturated ester monomer(s) of acrylate or methacrylate type having, in its or their structure, at least one alkyl group having a fatty chain is or are present in a content ranging from 0.1 to 5% by weight, preferably from 0.5 to 3% by weight and better still from 1 to 2% by weight, with respect to the total weight of the copolymer.

The acrylic copolymer also results from at least one unsaturated ester monomer of acrylate or methacrylate type having, in its structure, at least one short-chain alkyl group. These monomers are thus esters of acrylic or methacrylic acid and of a short-chain monoalcohol, the monoalcohol providing the alkyl group of the ester. The alkyl group can be linear or branched, preferably branched. Preferably, the unsaturated ester monomer of acrylate or methacrylate type having, in its structure, a short-chain alkyl group is an alkyl acrylate or methacrylate with a C₂-C₆, more preferably C₃-0₅ and better still C₄ alkyl group.

According to a preferred embodiment, the acrylic copolymer comprises, as monomers, a tert-butyl acrylate or methacrylate.

Preferably, the unsaturated ester monomer(s) of acrylate or methacrylate type having, in its or their structure, at least one short-chain alkyl group is or are present in a content ranging from 50 to 95% by weight, preferably from 60 to 90% by weight and better still from 70 to 85% by weight, with respect to the total weight of the copolymer.

The acrylic copolymer also comprises at least one acrylamide monomer substituted by at least one alkyl group. The alkyl group can be linear or branched, preferably branched. Preferably, the alkyl group is a C₃-C₁₀, more preferably still a C₄-C₈, alkyl group.

According to a preferred embodiment, the acrylic copolymer comprises, as monomers, tert-butylacrylamide or tert-octylacrylamide.

More preferably still, the acrylic copolymer comprises, as monomers, tert-butylacrylamide and tert-octylacrylamide.

Preferably, the acrylamide monomer(s) substituted by at least one alkyl group is or are present in a content ranging from 0.01 to 10% by weight, preferably from 1 to 5% by weight, with respect to the weight of the copolymer.

The acrylic copolymer can additionally comprise, as monomers, one or more hydroxylated ester of acrylic or methacrylic acid monomers. Preferably, the ester is a hydroxylated C₂-C₆, more preferably C₂, alkyl acrylate or methacrylate, such as hydroxyethyl acrylate or methacrylate.

The acrylic copolymer can have an average molecular weight ranging from 20 000 to 500 000 g/mol, preferably from 100 000 to 250 000 g/mol.

Preferably, the copolymer in accordance with the invention is in the salt form by the neutralization of the acid residues resulting from the acrylic acid or methacrylic acid. The copolymer can be partially or completely neutralized by an inorganic base (sodium hydroxide, potassium hydroxide or aqueous ammonia) or an organic base, such as monoethanolamine, diethanolamine or triethanolamine, an aminomethylpropanol, N-methylglucamine, basic amino acids, such as arginine and lysine, and the mixtures of these compounds. Preferably, the acrylic copolymer is neutralized by a neutralizing agent chosen from aminomethylpropanol, triethanolamine

Preferably, the degree of neutralization of the acrylic copolymer varies from 50 to 100%, preferably from 80 to 100%, and more particularly the acrylic copolymer is 100% neutralized.

Use will be made, for example, as acrylic copolymer according to the invention, of an AMP-acrylates/C₁-C₁₈ alkyl acrylate/C₁-C₈ alkylacrylamide/hydroxyethyl acrylate copolymer sold by GOO Chemical under the commercial reference Plascize L-9700 or Plascize L-9700U.

The acrylic copolymer can be present in the composition in a content ranging from 0.5 to 20% by weight, preferably from 1 to 10% by weight, with respect to the total weight of the composition.

As indicated above, the composition according to the invention also comprises one or more solvents other than 1,1-difluoroethane.

The solvent can be chosen from water, an organic solvent or a mixture of water and of one or more organic solvents, which are preferably water-soluble.

The term “organic solvent” is understood to mean, within the meaning of the present invention, an organic compound which is liquid at a temperature of 25° C. and at atmospheric pressure (760 mm Hg).

The term “water-soluble” is understood to mean, within the meaning of the present invention, a compound which is soluble to at least 5% by weight in water at a temperature of 25° C. and at atmospheric pressure (760 mm Hg).

Preferably, the organic compound is polar.

The organic solvents can be chosen from short chain, for example C₁-C₄, monoalcohols, such as ethanol or isopropanol; diols or polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether and sorbitol.

In one alternative form of the invention, the composition comprises water.

In this alternative form, the composition does or does not comprise one or more water-soluble organic solvents, such as ethanol or isopropanol.

In another alternative form of the invention, the composition does not comprise water.

The composition according to the invention can comprise one or more solvents other than 1,1-difluoroethane in an amount ranging from 30 to 90%, preferably from 40 to 80%, by weight, with respect to the total weight of the composition.

The composition according to the invention additionally comprises a propellant comprising 1,1-difluoroethane.

The at least one of the constituents of the propellant is 1,1-difluoroethane.

In an alternative form of the invention, the propellant consists solely of 1,1-difluoroethane.

In another alternative form of the invention, the propellant is composed of 1,1-difluoroethane in combination with one or more other constituents preferably chosen from:
volatile hydrocarbons, such as in particular C₁ to C₄ alkanes and preferably n-butane, propane, isobutane and their mixtures;
chlorinated and/or fluorinated hydrocarbons other than 1,1-difluoroethane;
dimethyl ether;
and the mixtures of these constituents.

Preferably, the composition according to the invention comprises solely 1,1-difluoroethane as propellant.

This compound is sold in particular under the name Dymel 152-A by DuPont.

The composition according to the invention preferably comprises 1,1-difluoroethane in a content ranging from 20 to 60% by weight, better still from 25 to 55% by weight, with respect to the total weight of the composition according to the invention.

The composition according to the invention can additionally comprise one or more surfactants which can in particular be chosen from non-ionic, cationic, anionic and amphoteric or zwitterionic surface-active agents.

The said surface-active agent(s) according to the invention preferably comprise one or more non-ionic surface-active agent(s).

The non-ionic surface-active agent(s) which can be used in the present composition are described, for example, in “Handbook of Surfactants” by M. R. Porter, published by Blackie & Son (Glasgow and London), 1991, pp. 116-178.

Mention may be made, as examples of non-ionic surface-active agents, of the following non-ionic surface-active agents:
oxyalkylenated (C₈-C₂₄)alkylphenols;
saturated or unsaturated, linear or branched, oxyalkylenated or glycerolated C₄-C₄₀ alcohols;
saturated or unsaturated, linear or branched, oxyalkylenated C₈-C₃₀ fatty acid amides;
esters of saturated or unsaturated, linear or branched, C₈-C₃₀ acids and of polyethylene glycols;
esters of saturated or unsaturated, linear or branched, C₈-C₃₀ acids and of sorbitol, which are preferably oxyethylenated;
esters of fatty acids and of sucrose;
   (C₈-C₃₀)alkyl(poly)glucosides, (C₈-C₃₀)alkenyl(poly)glucosides, which are optionally oxyalkylenated (0 to 10 oxyalkylene units) and comprising from 1 to 15 glucose units, (C₈-C₃₀)alkyl (poly)glucoside esters;
saturated or unsaturated oxyethylenated vegetable oils;
condensates of ethylene oxide and/or of propylene oxide, inter alia, alone or as mixtures;
N-(C₈-C₃₀)alkylglucamine and N-(C₈-C₃₀)acylmethylglucamine derivatives;
aldobionamides;
amine oxides;
oxyethylenated and/or oxpropylenated silicones;
and their mixtures.

The oxyalkylene units are more particularly oxyethylene or oxypropylene units, or their combination, preferably oxyethylene units.

The number of moles of ethylene oxide and/or of propylene oxide preferably ranges from 1 to 250, more particularly from 2 to 100 and better still from 2 to 50; the number of moles of glycerol ranges in particular from 1 to 50 and better still from 1 to 10.

Advantageously, the non-ionic surface-active agents according to the invention do not comprise oxpropylene units.

Use is preferably made, as examples of glycerolated non-ionic surface-active agents, of monoglycerolated or polyglycerolated C₈-C₄₀ alcohols, comprising from 1 to 50 mol of glycerol and preferably from 1 to 10 mol of glycerol.

Mention may be made, as examples of compounds of this type, of lauryl alcohol comprising 4 mol of glycerol (INCI name: Polyglyceryl-4 Lauryl Ether), lauryl alcohol comprising 1.5 mol of glycerol, oleyl alcohol comprising 4 mol of glycerol (INCI name: Polyglyceryl-4 Oleyl Ether), oleyl alcohol comprising 2 mol of glycerol (INCI name: Polyglyceryl-2 Oleyl Ether), cetearyl alcohol comprising 2 mol of glycerol, cetearyl alcohol comprising 6 mol of glycerol, oleocetyl alcohol comprising 6 mol of glycerol and octadecanol comprising 6 mol of glycerol.

Preference is more particularly given, among the glycerolated alcohols, to the use of the C₈/C₁₀ alcohol comprising 1 mol of glycerol, the C₁₀/C₁₂ alcohol comprising 1 mol of glycerol and the C₁₂ alcohol comprising 1.5 mol of glycerol.

The non-ionic surface-active agent(s) according to the invention are preferentially chosen from:
oxyethylenated C₄-C₄₀ alcohols comprising from 1 to 100 mol of ethylene oxide, preferably from 2 to 50 and more particularly from 2 to 40 mol of ethylene oxide;
saturated or unsaturated oxyethylenated vegetable oils comprising from 1 to 100 and preferably from 2 to 50 mol of ethylene oxide;
(C₈-C₃₀)alkyl(poly)glucosides, which are optionally oxyalkylenated (0 to 10 OE) and comprising from 1 to 15 glucose units;
monoglycerolated or polyglycerolated C₈-C₄₀ alcohols, comprising from 1 to 50 mol of glycerol and preferably from 1 to 10 mol of glycerol;
saturated or unsaturated, linear or branched, oxyalkylenated C₈-C₃₀ fatty acid amides;
esters of saturated or unsaturated, linear or branched, C₈-C₃₀ acids and of polyethylene glycols;
and their mixtures.

More preferably still, the non-ionic surface-active agent(s) according to the invention are chosen from:
saturated or unsaturated, linear or branched, oxyalkylenated C₄-C₄₀ alcohols, in particular PPG-7-Buteth-10;
(C₈-C₃₀)alkyl(poly)glucosides, in particular caprylyl/capryl glucoside;
esters of saturated or unsaturated, linear or branched, C₈-C₃₀ acids and of polyethylene glycols and in particular PEG-8 isostearate;
and their mixtures.

The composition according to the invention can comprise one or more cationic surface-active agent(s).

The term “cationic surface-active agent” is understood to mean a surface-active agent which is positively charged when it is present in the composition according to the invention. This surface-active agent can carry one or more positive permanent charges or can comprise one or more cationizable functional groups within the composition according to the invention.

The cationic surface-active agent(s) are preferably chosen from optionally polyoxyalkylenated primary, secondary or tertiary fatty amines, or their salts, quaternary ammonium salts, and their mixtures.

The fatty amines generally comprise at least one C₈-C₃₀ hydrocarbon chain.

Mention may in particular be made, as quaternary ammonium salts, for example, of:
those corresponding to the following general formula (I):

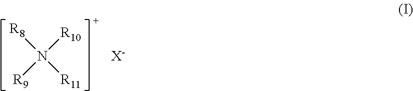

in which the groups R₈ to R₁₁, which can be identical or different, represent a linear or branched aliphatic group comprising from 1 to 30 carbon atoms, or an aromatic group, such as aryl or alkylaryl, at least one of the groups R₈ to R₁₁ denoting a group comprising from 8 to 30 carbon atoms and preferably from 12 to 24 carbon atoms. The aliphatic groups can comprise heteroatoms, such as, in particular, oxygen, nitrogen, sulfur and halogens. The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of the halides, phosphates, acetates, lactates, (C₁-C₄)alkyl sulfates and (C₁-C₄)alkyl- or (C₁-C₄)alkylarylsulfonates.

Preference is given, among the quaternary ammonium salts of formula (I), on the one hand, to tetraalkylammonium salts, such as, for example, dialkyldimethylammonium or alkyltrimethylammonium salts, in which the alkyl group comprises approximately from 12 to 22 carbon atoms, in particular behenyltrimethylammonium, distearyldimethylammonium, cetyltrimethylammonium or benzyldimethylstearylammonium salts, or also, on the other hand, to palmitylamidopropyltrimethylammonium salts, stearamidopropyltrimethylammonium salts, stearamidopropyldimethylcetearylammonium salts or stearamidopropyldimethyl(myristyl acetate)ammonium salts sold under the name Ceraphyl® 70 by Van Dyk. It is preferable in particular to use the chloride salts of these compounds.
quaternary ammonium salts of imidazoline, such as, for example, those of following formula (II):

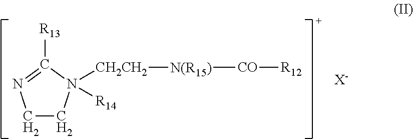

in which R₁₂ represents an alkenyl or alkyl group comprising from 8 to 30 carbon atoms, for example derived from tallow fatty acids, R₁₃ represents a hydrogen atom, a C₁-C₄ alkyl group or an alkenyl or alkyl group comprising from 8 to 30 carbon atoms, R₁₄ represents a C₁-C₄ alkyl group, R₁₅ represents a hydrogen atom or a C₁-C₄ alkyl group and X⁻ is an anion chosen from the group of the halides, phosphates, acetates, lactates, alkyl sulfates, alkylsulfonates or alkylarylsulfonates, the alkyl and aryl groups of which preferably comprise, respectively, from 1 to 20 carbon atoms and from 6 to 30 carbon atoms. Preferably, R₁₂ and R₁₃ denote a mixture of alkenyl or alkyl groups comprising from 12 to 21 carbon atoms, for example derived from tallow fatty acids, R₁₄ denotes a methyl group and R₁₅ denotes a hydrogen atom. Such a product is sold, for example, under the name Rewoquat® W 75 by Rewo.
di- or triquaternary ammonium salts, in particular of formula (III):

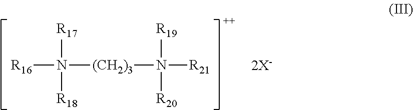

in which R₁₆ denotes an alkyl radical comprising approximately from 16 to 30 carbon atoms which is optionally hydroxylated and/or interrupted by one or more oxygen atoms, R₁₇ is chosen from hydrogen or an alkyl radical comprising from 1 to 4 carbon atoms or an (R₁₆ₐ)(R₁₇ₐ)(R_{18a)N}⁺(CH₂)₃-group, R₁₆ₐ, R₁₇ₐ, R₁₈ₐ, R₁₈, R₁₉, R₂₀ and R₂₁, which are identical or different, are chosen from hydrogen or an alkyl radical comprising from 1 to 4 carbon atoms, and X⁻ is an anion chosen from the group of the halides, acetates, phosphates, nitrates and methyl sulfates. Such compounds are, for example, Finquat CT-P, provided by the company Finetex (Quaternium 89), and Finquat CT, provided by the company Finetex (Quaternium 75).
quaternary ammonium salts comprising at least one ester functional group, such as those of following formula (IV):

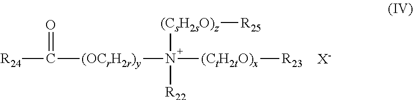

in which:

R₂₂ is chosen from C₁-C₆ alkyl groups and C₁-C₆ hydroxyalkyl or dihydroxyalkyl groups;

R₂₃ is chosen from:
the group

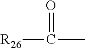

R₂₇ groups, which are saturated or unsaturated, linear or branched, C₁-C₂₂ hydrocarbon groups,
a hydrogen atom,

R₂₅ is chosen from:
the group

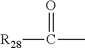

R₂₉ groups, which are saturated or unsaturated, linear or branched, C₁-C₆ hydrocarbon groups,
a hydrogen atom,

R₂₄, R₂₆ and R₂₈, which are identical or different, are chosen from saturated or unsaturated, linear or branched, C₇-C₂₁ hydrocarbon groups;

s and t, which are identical or different, are integers having values from 2 to 6;

y is an integer having a value from 1 to 10;

x and z, which are identical or different, are integers having a value from 0 to 10;

X⁻ is a simple or complex and organic or inorganic anion;

with the proviso that the sum x +y +z has a value from 1 to 15, that, when x has the value 0, then R₂₃ denotes R₂₇ and that, when z has the value 0, then R₂₅ denotes R₂₉.

The R₂₂ alkyl groups can be linear or branched and more particularly linear.

Preferably, R₂₂ denotes a methyl, ethyl, hydroxyethyl or dihydroxypropyl group and more particularly a methyl or ethyl group.

Advantageously, the sum x+y+z has a value from 1 to 10.

When R₂₃ is an R₂₇ hydrocarbon group, it can be long and have from 12 to 22 carbon atoms or be short and have from 1 to 3 carbon atoms.

When R₂₅ is an R₂₉ hydrocarbon group, it preferably has from 1 to 3 carbon atoms.

Advantageously, R₂₄, R₂₆ and R₂₈, which are identical or different, are chosen from saturated or unsaturated, linear or branched, C₁₁-C₂₁ hydrocarbon groups and more particularly from saturated or unsaturated, linear or branched, C₁₁-C₂₁ alkyl and alkenyl groups.

Preferably, x and z, which are identical or different, have the value 0 or 1.

Advantageously, y is equal to 1.

Preferably, r, s and t, which are identical or different, have the value 2 or 3 and more particularly still are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion which is compatible with the ammonium having an ester functional group.

The anion X⁻ is more particularly still chloride or methyl sulfate.

Use is more particularly made, in the composition according to the invention, of the ammonium salts of formula (IV) in which:

R₂₂ denotes a methyl or ethyl group,

x and y are equal to 1;

z is equal to 0 or 1;

s and t are equal to 2;

R₂₃ is chosen from:
the group

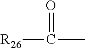

methyl, ethyl or C₁₄-C₂₂ hydrocarbon groups,
a hydrogen atom;

R₂₅ is chosen from:
the group

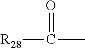

a hydrogen atom;

R₂₄, R₂₆ and R₂₈, which are identical or different, are chosen from saturated or unsaturated, linear or branched, C₁₃-C₁₇ hydrocarbon groups and preferably from saturated or unsaturated, linear or branched, C₁₃-C₁₇ alkyl and alkenyl groups.

Advantageously, the hydrocarbon groups are linear.

Mention may be made, for example, of compounds of formula (IV), such as diacyloxyethyldimethylammonium, diacyloxyethyl(hydroxyethyl)methylammonium, monoacyloxyethyldi(hydroxyethyl)methylammonium, triacyloxyethylmethylammonium or monoacyloxyethyl(hydroxyethyl)dimethylammonium salts (in particular chloride or methyl sulfate) and their mixtures. The acyl groups preferably have from 14 to 18 carbon atoms and originate more particularly from a vegetable oil, such as palm oil or sunflower oil. When the compound comprises several acyl groups, the latter can be identical or different.

These products are obtained, for example, by direct esterification of triethanolamine, triisopropanolamine, alkyldiethanolamine or alkyldiisopropanolamine, which are optionally oxyalkylenated, with C₁₀-C₃₀ fatty acids or with mixtures of C₁₀-C₃₀ fatty acids of vegetable or animal origin, or by transesterification of their methyl esters. This esterification is followed by a quaternization using an alkylating agent, such as an alkyl halide (preferably methyl halide or ethyl halide), a dialkyl sulfate (preferably dimethyl sulfate or diethyl sulfate), methyl methanesulfonate, methyl pare-toluenesulfonate, glycol chlorohydrin or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the invention can comprise, for example, a mixture of quaternary ammonium mono-, di- and triester salts with a predominance by weight of diester salts.

Use may also be made of the ammonium salts comprising at least one ester functional group which are described in Patents U.S. Pat. No. 4,874,554 and U.S. Pat. No. 4,137,180.

Use may be made of behenoylhydroxypropyltrimethylammonium chloride, provided by Kao under the name Quatarmin BTC 131.

Preferably, the ammonium salts comprising at least one ester functional group comprise two ester functional groups.

Preference is given, among the quaternary ammonium salts comprising at least one ester functional group which can be used, to the use of dipalmitoylethylhydroxyethylmethylammonium salts.

The cationic surface-active agents are preferably chosen from those of formula (I) and those of formula (IV) and more preferably still from those of formula (I).

The composition according to the invention can comprise one or more anionic surface-active agent(s).

The term “anionic surface-active agent” is understood to mean a surface-active agent comprising, as ionic or ionizable groups, only anionic groups. These anionic groups are preferably chosen from the following groups: —COOH, —COO⁻, —SO₃H, —SO₃⁻, —OSO₃H, —OSO₃⁻, —PO₂H₂, —PO₂²⁻, —P(OH)₂), ═P(O)OH, —P(OH)O⁻, ═P(O)O⁻, ═POH, ═PO⁻, the anionic parts comprising a cationic counterion, such as an alkali metal, an alkaline earth metal or an ammonium.

Mention may be made, as examples of anionic surface-active agents which can be used in the composition according to the invention, of alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefinsulfonates, paraffinsulfonates, alkyl sulfosuccinates, alkyl ether sulfosuccinates, alkylamide sulfosuccinates, alkyl sulfoacetates, acyl sarcosinates, acyl glutamates, alkyl sulfosuccinamates, acyl isethionates and N-acyl taurates, salts of alkyl monoesters of polyglucoside-polycarboxylic acids, acyl lactylates, salts of D-galactosideuronic acids, salts of alkyl ether carboxylic acids, salts of alkylaryl ether carboxylic acids, salts of alkylamido ether carboxylic acids; alkyl or alkenyl phosphates, and the corresponding non-salified forms of all these compounds, the alkyl and acyl groups of all these compounds comprising from 6 to 40 carbon atoms and the aryl group denoting a phenyl group.

These compounds can be oxyethylenated and then preferably comprise from 1 to 50 ethylene oxide units.

The salts of C₆-C₂₄ alkyl monoesters of polyglucoside-polycarboxylic acids can be chosen from C₆-C₂₄ alkyl polyglucoside-citrates, C₆-C₂₄ alkyl polyglucoside-tartrates and C₆-C₂₄ alkyl polyglucoside-sulfosuccinates.

When the anionic surface-active agent(s) are in the salt form, they can be chosen from alkali metal salts, such as the sodium or potassium salt and preferably the sodium salt, ammonium salts, amine salts and in particular amino alcohol salts, or alkaline earth metal salts, such as the magnesium salts.

Mention may in particular be made, as examples of amino alcohol salts, of mono-, di- and triethanolamine salts, mono-, di- or triisopropanolamine salts, 2-amino-2-methyl-1-propanol salts, 2-amino-2-methyl-1,3-propanediol salts and tris(hydroxymethyl)aminomethane salts.

Use is preferably made of alkali metal or alkaline earth metal salts and in particular of sodium or magnesium salts.

Use is preferably made, among the anionic surface-active agents mentioned, of (C₆-C₂₄)alkyl sulfates, (C₆-C₂₄)alkyl ether sulfates, (C₆-C₂₄)alkyl or (C₆-C₂₄)alkenyl phosphates comprising from 2 to 50 ethylene oxide units, in particular in the form of alkali metal, ammonium, amino alcohol and alkaline earth metal salts, or a mixture of these compounds.

In particular, use is preferably made of (C₁₂-C₂₀)alkyl sulfates, (C₁₂-C₂₀)alkyl ether sulfates, (C₁₂-C₂₄)alkyl or (C₁₂-C₂₄)alkenyl phosphates comprising from 2 to 20 ethylene oxide units, in particular in the form of alkali metal, ammonium, amino alcohol and alkaline earth metal salts, or a mixture of these compounds. Better still, use is preferably made of oleth-20 phosphate.

The composition according to the invention can comprise one or more amphoteric or zwitterionic surface-active agent(s).

In particular, the amphoteric or zwitterionic surfactant(s), which are preferably non-silicone, which can be used in the present invention can in particular be derivatives of optionally quaternized aliphatic secondary or tertiary amines, in which derivatives the aliphatic group is a linear or branched chain comprising from 8 to 22 carbon atoms, the said amine derivatives comprising at least one anionic group, such as, for example, a carboxylate, sulfonate, sulfate, phosphate or phosphonate group.

Mention may in particular be made of (C₈-C₂₀)alkyl betaines, (C₈-C₂₀)alkyl sulfobetaines, (C₈-C₂₀)alkylamido (C₃-C₈)alkyl betaines and (C₈-C₂₀)alkylamido (C₆-C₈)alkyl sulfobetaines.

Mention may also be made, among the optionally quaternized aliphatic secondary or tertiary amine derivatives which can be used, as defined above, of the compounds having the following respective structures (V) and (VI):

Rₐ—CONHCH₂CH₂—N⁺(R_{b})(R_{c})—CH₂COO⁻M⁺X⁻ (V)

in which formula:
Rₐ represents a C₁₀-C₃₀ alkyl or alkenyl group derived from an acid Rₐ—COOH preferably present in hydrolysed coconut oil, or a heptyl, nonyl or undecyl group;
   R_{b} represents a 8-hydroxyethyl group; and
   R_{c} represents a carboxymethyl group;
M⁺ represents a cationic counterion resulting from an alkali metal or alkaline earth metal, such as sodium, an ammonium ion or an ion resulting from an organic amine; and
   X⁻ represents an organic or inorganic anionic counterion, such as that chosen from halides, acetates, phosphates, nitrates, (C₁-C₄)alkyl sulfates and (C₁-C₄)alkyl- or (C₁-C₄)alkylarylsulfonates, in particular methyl sulfate and ethyl sulfate; or alternatively M⁺ and X⁻ are absent;

Rₐ′—CONHCH₂CH₂—N(B)(B′) (VI)

in which formula:
B represents the group —CH₂CH₂OX′;
B represents the group —(CH₂),Y′, with z=1 or 2;
X represents the group —CH₂COOH, —CH₂—COOZ′,

—CH₂CH₂COOH or —CH₂CH₂-COOZ', or a hydrogen atom;
Y′ represents the group —COOH, —COOZ′ or

CH₂CH(OH)SO₃H or the group CH₂CH(OH)SO₃—Z′;
Z′ represents a cationic counterion resulting from an alkali metal or alkaline earth metal, such as sodium, an ammonium ion or an ion resulting from an organic amine;
Rₐ′ represents a C₁₀-C₃₀ alkyl or alkenyl group of an acid Rₐ′—COOH preferably present in hydrolysed linseed oil or coconut oil, an alkyl group, in particular a C₁₇ alkyl group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of example, of the cocoamphodiacetate sold by Rhodia under the trade name Miranol® C2M Concentrate.

Use may also be made of compounds of formula (VII):

Rₐ″—NHCH(Y″)—(CH₂)ₙCONH(CH₂)ₙ—N(R_{d})(Rₑ) (VII)

in which formula:
Y″ represents the group —COOH, —COOZ″ or —CH₂—CH(OH)SO₃H or the group CH₂CH(OH)SO₃—Z″;
R_{d} and Rₑ represent, independently of each other, a C₁-C₄ alkyl or hydroxyalkyl radical;
Z″ represents a cationic counterion resulting from an alkali metal or alkaline earth metal, such as sodium, an ammonium ion or an ion resulting from an organic amine;
Rₐ″ represents a C₁₀-C₃₀ alkyl or alkenyl group of an acid Rₐ″—COOH preferably present in hydrolysed linseed oil or coconut oil;
n and n′ denote, independently of each other, an integer ranging from 1 to 3.

Mention may be made, among the compounds of formula (VII), of the compound classified in the CTFA dictionary under the name sodium diethylaminopropyl cocoaspartamide and sold by Chimex under the name Chimexane HB.

These compounds can be used alone or as mixtures.

Use is preferably made, among the abovementioned amphoteric or zwitterionic surface-active agents, of (C₈-C₂₀)alkyl betaines, such as cocoyl betaine, (C₈-C₂₀)alkylamido (C₃-C₈)alkyl betaines, such as cocamidopropyl betaine, and their mixtures, and of the compounds of formula (VII), such as the sodium salt of diethylaminopropyl laurylaminosuccinamate (INCI name: sodium diethylaminopropyl cocoaspartamide).

Preference is given, among all the surface-active agents mentioned, to the use of one or more non-ionic surface-active agent(s), preferably oxyethylenated, such as oxyethylenated C₄-C₄₀ alcohols comprising from 1 to 100 mol of ethylene oxide, preferably from 2 to 50 and more particularly from 2 to 40 mol of ethylene oxide, and phosphate-comprising anionic surfactants, such as (C₈-C₂₄)alkyl or (C₈-C₂₄)alkenyl phosphates comprising from 2 to 50 ethylene oxide units.

When they are present, the surface-active agent(s) represent from 0.001 to 20%, preferably from 0.005 to 15% and better still from 0.01 to 10% by weight, with respect to the total weight of the composition.

The composition according to the invention can additionally comprise one or more additives chosen from fixing polymers other than the acrylic copolymers according to the invention, conditioning agents, such as cationic polymers, fatty substances, silicones, ceramides and pseudoceramides, vitamins and provitamins, including panthenol, water-soluble or fat-soluble sunscreens, pearlescent and opacifying agents, sequestering agents, solubilizing agents, antioxidants, hydroxy acids, penetration agents, fragrances, peptizing agents, amino acids and preservatives, alone or as a mixture, and any other additive conventionally used in the cosmetics field.

A person skilled in the art will take care to choose the optional additives and their amounts so that they do not interfere with the properties of the compositions of the present invention.

These additives can be present in the composition according to the invention in an amount ranging from 0 to 20% by weight, with respect to the total weight of the composition.

The compositions according to the invention are packaged in an aerosol device.

The present invention also relates to a method for the shaping and/or form retention of the hairstyle comprising the use of the composition as defined above.

In particular, the method for the shaping and/or form retention of the hairstyle comprises a stage of application, to human keratinous fibres, in particular the hair, dry or wet, of a composition as defined above sprayed from an aerosol device, to be or not to be rinsed off after an optional leave-on time or after optional drying.

Preferably, the composition according to the invention is not rinsed off.

The cosmetic composition according to the invention can thus be sprayed over dry or wet hair. Preferably, the cosmetic composition is applied to clean hair.

According to a specific embodiment of the invention, the application of the composition can be followed by drying at ambient temperature or at a temperature of greater than 40° C.

The drying can be carried out immediately after the application or after a leave-on time which can range from 1 minute to 30 minutes.

The present invention also relates to an aerosol device comprising a pressurized container containing the composition described above, the container being surmounted by an actuator. The container can be equipped with a valve. The actuator can be provided with one outlet orifice for the composition, in particular with a nozzle having a simple or turbulent-channel orifice, or with several outlet orifices, in particular three orifices.

Any type of container and valve system for an aerosol composition is appropriate for the implementation of the present invention.

The present invention also relates to the use of a composition as defined above for the shaping and/or form retention of the hairstyle.

The examples which follow serve to illustrate the invention without, however, exhibiting a limiting nature.

### EXAMPLE

The following composition A according to the invention was prepared from the ingredients indicated, in grams of active material, in the table below:

| Ingredients | Example A |
|---|---|
| AMP-acrylates/C₁-C₁₈ alkyl acrylate/C₁-C₈ | 4.4 |
| alkylacrylamide/hydroxyethyl acrylate copolymer⁽¹⁾ | |
| 1,1-Difluoroethane | 45 |
| Ethanol | q.s. for 100 |

⁽¹⁾Plascize L-9700U, sold by GOO Chemical (38% AMP-acrylates/C₁-C₁₈ alkyl acrylate/C₁-C₈ alkylacrylamide/hydroxyethyl acrylate copolymer, 1% Oleth-20 phosphate, 1% PPG-7-Buteth-10 and 60% ethanol)

The composition prepared above was introduced into an aerosol dispensing device.

A valve was used comprising:
a spray nozzle orifice of 0.33 mm,
no additional gas intake orifice,
an internal restriction orifice of 0.33 mm.

An actuator equipped with a turbulent-channel nozzle with an outlet orifice having a diameter of 0.45 mm was used.

The composition was vaporized over dry hair.

drying, a head of hair exhibiting significant long-lasting fixing is obtained. The feel is very satisfactory (softness, suppleness).

1-17. (canceled)

18. A composition comprising:
(i) at least one acrylic copolymer comprising:
(a) at least one unsaturated acid monomer chosen from acrylic acid, methacrylic acid, or salts thereof;
(b) at least one unsaturated ester monomer of acrylate or methacrylate chosen from esters comprising at least one alkyl group comprising a linear or branched fatty chain with 8 to 30 carbon atoms;
(c) at least one unsaturated ester monomer of acrylate or methacrylate chosen from esters comprising at least one alkyl group comprising a linear or branched short chain with 2 to 6 carbon atoms; and
(d) at least one acrylamide monomer comprising a linear or branched alkyl group with 3 to 10 carbon atoms;
(ii) at least one solvent; and
(iii) at least one propellant comprising 1,1-difluoroethane.

19. The composition according to claim 18, wherein at least one alkyl group is branched.

20. The composition according to claim 18, wherein the at least one acrylic copolymer further comprises at least one hydroxylated ester of acrylic or methacrylic acid monomers.

21. The composition according to claim 20, wherein the at least one hydroxylated ester is C2-C6 alkyl acrylate or C2-C6 alkyl methacrylate.

22. The composition according to claim 18, wherein the amount of the acrylic acid, the methacrylic acid, or salt thereof ranges from about 2% to about 10% by weight, relative to the weight of the copolymer.

23. The composition according to claim 18, wherein the amount of the at least one unsaturated ester monomer of acrylate or methacrylate comprising at least one alkyl group comprising a linear or branched fatty chain with 8 to 30 carbon atoms ranges from about 0.1% to about 5% by weight, relative to the total weight of the copolymer.

24. The composition according to claim 18, wherein the amount of the at least one unsaturated ester monomer of acrylate or methacrylate comprising at least one alkyl group comprising a linear or branched short chain with 2 to 6 carbon atoms ranges from about 50% to about 95% by weight, relative to the total weight of the copolymer.

25. The composition according to claim 18, wherein the amount of the at least one acrylamide monomer ranges from about 0.01% to about 10% by weight, relative to the weight of the copolymer.

26. The composition according to claim 18, wherein the molecular weight of the at least one acrylic copolymer ranges from about 20,000 g/mol to about 500,000 g/mol.

27. The composition according to claim 18, wherein the amount of the at least one acrylic copolymer ranges from about 0.5% to about 20% by weight, relative to the total weight of the composition.

28. The composition according to claim 18, further comprising at least one surfactant chosen from non-ionic, anionic, cationic, amphoteric or zwitterionic surfactants.

29. The composition according to claim 28, wherein the amount of the at least one surfactant ranges from about 0.001% to about 20% by weight, relative to the total weight of the composition.

30. The composition according to claim 28, wherein the at least one solvent is chosen from water, water-soluble organic solvents, or mixtures thereof.

31. The composition according to claim 28, wherein the amount of the at least one solvent ranges from about 30% to about 90% by weight, relative to the total weight of the composition.

32. The composition according to claim 18, wherein the amount of 1,1-difluoroethane ranges from about 20% to about 60% by weight, relative to the total weight of the composition.

33. The composition according to claim 18, wherein the propellant consists of 1,1-difluoroethane.

34. A method for shaping and/or form retention of the hairstyle comprising applying to the hair an effective amount of a composition, wherein the composition comprises:
(i) at least one acrylic copolymer comprising:
(a) at least one unsaturated acid monomer chosen from acrylic acid, methacrylic acid, or salts thereof;
(b) at least one unsaturated ester monomer of acrylate or methacrylate chosen from esters comprising at least one alkyl group comprising a linear or branched fatty chain with 8 to 30 carbon atoms;
(c) at least one unsaturated ester monomer of acrylate or methacrylate chosen from esters comprising at least one alkyl group comprising a linear or branched short chain of 2 to 6 carbon atoms; and
(d) at least one acrylamide monomer comprising a linear or branched alkyl group with 3 to 10 carbon atoms;
(ii) at least one solvent; and
(iii) at least one propellant comprising 1,1-difluoroethane.

35. An aerosol device comprising a pressurized container comprising an actuator, wherein the container comprises a composition comprising:
(i) at least one acrylic copolymer comprising:
(a) at least one unsaturated acid monomer chosen from acrylic acid, methacrylic acid, or salts thereof;
(b) at least one unsaturated ester monomer of acrylate or methacrylate chosen from esters comprising at least one alkyl group comprising a linear or branched fatty chain with 8 to 30 carbon atoms;
(c) at least one unsaturated ester monomer of acrylate or methacrylate chosen from esters comprising at least one alkyl group comprising a linear or branched short chain of 2 to 6 carbon atoms; and
(d) at least one acrylamide monomer comprising a linear or branched alkyl group with 3 to 10 carbon atoms;
(ii) at least one solvent; and
(iii) at least one propellant comprising 1,1-difluoroethane.
